(19) United States
(12) Patent Application Publication
(10) Pub. No.: US 2011/0106242 A1
(43) Pub. Date: May 5, 2011

(54) ARTIFICIAL BLOOD VESSEL

(76) Inventors: Po-Jen Ko, Taipei City (TW); Yun-Hen Liu, Guishan Shiang (TW); Ching-Yang Wu, Linkou Township (TW)

(21) Appl. No.: 12/608,111

(22) Filed: Oct. 29, 2009

(51) Int. Cl. A61F 2/06

(52) U.S. Cl. 623/1.44

(57) ABSTRACT

An artificial blood vessel includes an artificial blood vessel body layer and a silicone layer. The artificial blood vessel body layer comprises an injection section, and the silicone layer is covered and mounted on an outer surface of the injection section. The artificial blood vessel body layer and the silicone layer are formed integrally by injection-molding. The silicone layer covered and mounted on the injection section of the artificial blood vessel body layer enables the artificial blood vessel for repeated injections and effectively prevents puncture aneurysm from occurring.

### BACKGROUND OF THE INVENTION

1. Field of the Invention

The present invention relates to an artificial blood vessel, and more particularly to an artificial blood vessel having a silicone layer covered and mounted on an outer surface of an injection section for repeated injections.

2. Description of Related Art

Referring to FIGS. 7-9, a conventional artificial blood vessel includes an artificial blood vessel body layer 50 for a needle 60 injection. The needle 60 has a bevel and sharp end 61. After inserting the sharp end 61 into a skin layer 70 and then puncturing into the artificial blood vessel body layer 50, the injection is finished.

However, after conventional needle punctures, the artificial blood vessel body layer 50 will have a hole 51 and a clot 52 resulted from the puncturing. The hole 51 will be easily filled with the blood generated from the puncturing. Therefore, with respect to the conventional artificial blood vessel, the injection wound has to be pressed after injection in order to prevent blood from flowing into the artificial blood vessel.

Moreover, the conventional artificial blood vessel has a short lifetime due to repeated injections, especially the part that needs to be often injected. Therefore, a replacement of the artificial blood vessel is frequently required. Thus, it is very inconvenient. Besides, with respect to the conventional artificial blood vessel, it cannot be reused right after one injection to another because it has to wait until the blood coagulates and falls off prior to a next injection. Thus, the need for improvement still exists.

### SUMMARY OF THE INVENTION

It is therefore one object of the invention to provide an artificial blood vessel, comprising: an artificial blood vessel body layer comprising at least one injection section and a silicone layer covered and mounted on an outer surface of the injection section.

In the first aspect of the invention, the artificial blood vessel further comprises two injection sections.

In the second aspect of the invention, the artificial blood vessel further comprises an obtuse needle.

In the third aspect of the invention, the obtuse needle further comprises an injection end, which is bended.

In the fourth aspect of the invention, the artificial blood vessel body layer is an injection-molding configuration.

In the fifth aspect of the invention, the artificial blood vessel body layer and the silicone layer are formed integrally into an injection-molding configuration.

By utilizing the invention, the following advantages are obtained. First, the present invention provides a silicone layer covered and mounted on the injection section of the artificial blood vessel body layer to enable the artificial blood vessel for repeated injections and prevent puncture aneurysm from occurring. The present invention can be re-used right away when it has been just used. The puncturing hole of the silicone layer generated from the puncturing can be re-closed right away. Thus, the present invention is very practical and convenient.

Second, the present invention mainly combines the design of the obtuse needle and the silicone layer. The present invention utilizes the obtuse needle to obtain a smaller puncturing hole. After the silicone layer is punctured, only a tiny puncturing point appears on the silicone layer and will be re-closed soon. Thus, the artificial blood vessel in accordance with the present invention has a longer lifetime and is more durable. It also effectively prevents the blood from flowing into the artificial blood vessel and thus prolongs the lifetime of the artificial blood vessel.

Third, the present invention utilizes a silicone layer covered and mounted on the injection section of the artificial blood vessel body layer to obtain the thicker and stronger injection section of the artificial blood vessel. Thus, the artificial blood vessel is not only mounted in the skin layer tightly but also effectively prevents the blood from flowing into the artificial blood vessel.

Further benefits and advantages of the present invention will become apparent after a careful reading of the detailed description with appropriate reference to the accompanying drawings.

### BRIEF DESCRIPTION OF THE DRAWINGS

FIG. 1 is a sectional view of an artificial blood vessel in accordance with the present invention;

FIG. 2 is another sectional view of an artificial blood vessel in accordance with the present invention;

FIG. 2A is a sectional view of an integrally formed artificial blood vessel in accordance with the present invention;

FIG. 3 is a perspective view of an artificial blood vessel mounted in a forearm of a patient according to one preferred embodiment of the present invention;

FIG. 4 is a perspective view of an artificial blood vessel mounted in an upper arm of a patient according to another preferred embodiment of the present invention;

FIG. 5 is a perspective view of an artificial blood vessel according to another preferred embodiment of the present invention;

FIG. 6 is a perspective view of an obtuse needle in accordance with the present invention;

FIG. 7 is a sectional view of a conventional artificial blood vessel being inserted by a conventional needle;

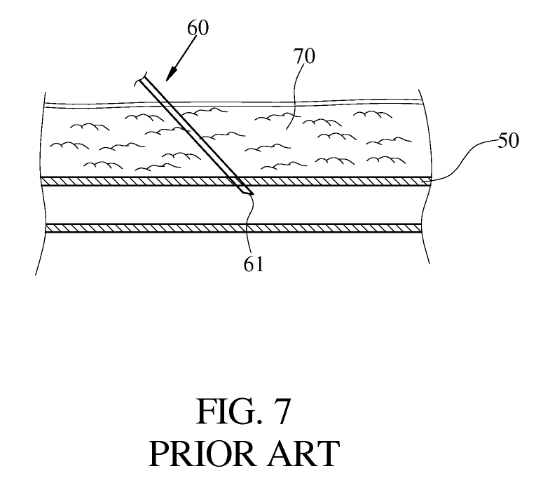

FIG. 8 is a sectional view showing the conventional needle being removed out of the conventional artificial blood vessel; and

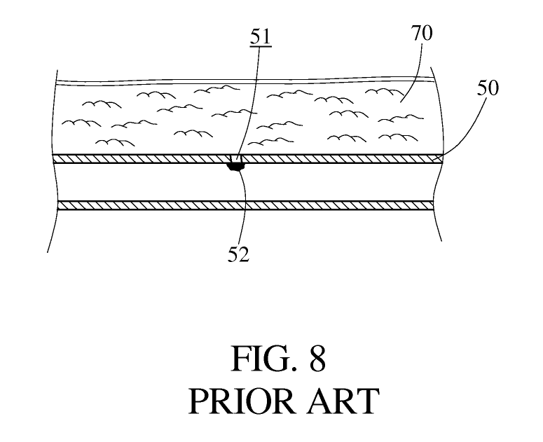

FIG. 9 is a perspective view of the conventional needle.

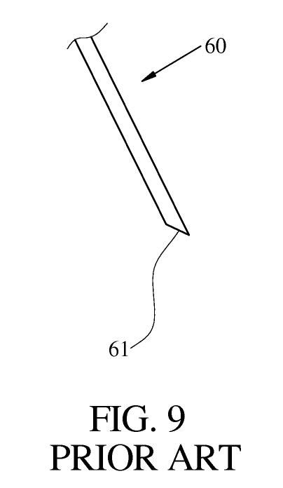

### DETAILED DESCRIPTION OF THE INVENTION

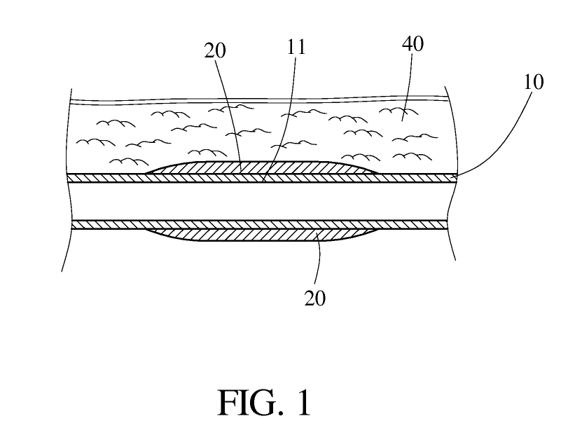

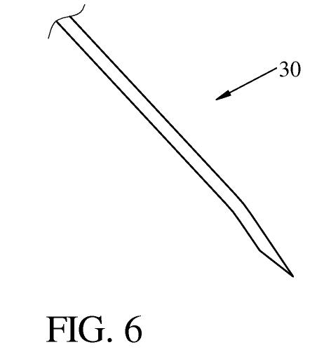

Referring to FIGS. 1-6, an artificial blood vessel 1 comprises an artificial blood vessel body layer 10 and a silicone layer 20. The artificial blood vessel body layer 10 comprises at least one injection section 11 and the silicone layer 20 is covered and mounted on an outer surface of the injection section 11. The silicone layer 20 can be made of a material similar to that of an injection top diaphragm of a port A. The material of the silicone layer 20 is capable of being injected repeatedly with a smaller puncturing hole. The puncturing hole can be re-closed. Thus, the improved artificial blood vessel 1 in accordance with the present invention is more durable than the conventional artificial blood vessel. The artificial blood vessel 1 is mounted under a skin layer 40 of a patient and is advantageous for repeated injections.

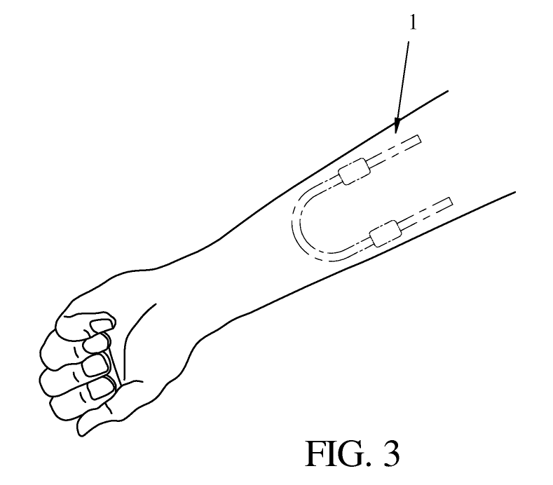

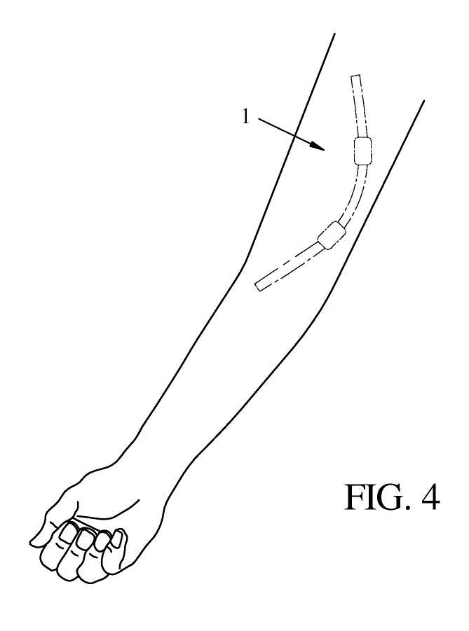

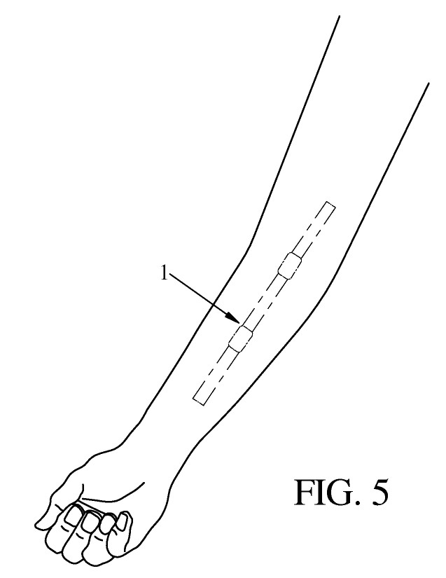

Preferably, the artificial blood vessel body layer 10 further comprises two injection sections 11. The artificial blood vessel 1 is U-shaped (as shown in FIG. 3). The two injection sections 11 are respectively mounted on two sides of the artificial blood vessel 1. Nevertheless, the artificial blood vessel 1 can be straight and mounted in the forearm or upper arm (as shown in FIGS. 4-5).

Preferably, the artificial blood vessel 1 further comprises an obtuse needle 30. The obtuse needle 30 is used for inserting through the silicone layer 20.

Preferably, the obtuse needle 30 further comprises a bent injection end. After the silicone layer 20 is punctured by the obtuse needle 30, only one tiny point appears on the silicone layer 20. The tiny point will be sealed quickly and, thus, it is re-closed and ready for re-use. Thus, the artificial blood vessel 1 in accordance with the present invention has a prolonged lifetime and is more durable. It also effectively prevents the blood from flowing into the artificial blood vessel 1.

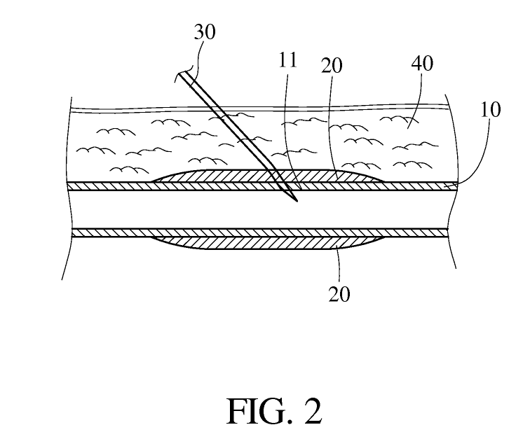

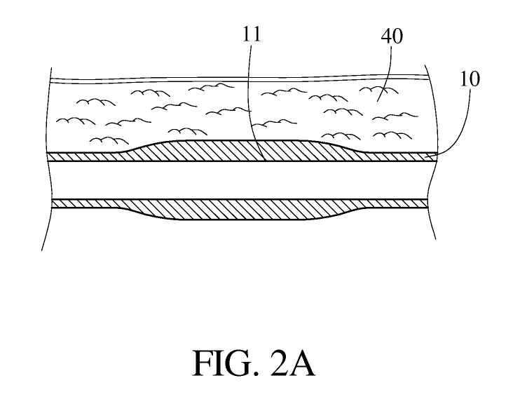

Preferably, the artificial blood vessel body layer 10 is formed by injection-molding. When the artificial blood vessel body layer 10 is injection molded, its temperature is very high and, therefore, the silicone layer 20 is connected to and melted with the artificial blood vessel body layer 10 (as shown in FIG. 2).

Preferably, the artificial blood vessel body layer 10 and the silicone layer 20 are formed integrally by injection-molding (as shown in FIG. 2A). Thus, it is very convenient to manufacture the artificial blood vessel 1 as contemplated by the present invention.

By utilizing the invention, the following advantages are obtained. First, the present invention provides a silicone layer covered and mounted on the injection section of the artificial blood vessel body layer to enable the artificial blood vessel for repeated injections. It can effectively prevent puncture aneurysm from occurring. The artificial blood vessel of the present invention can be reused immediately after being used. The puncturing hole of the silicone layer generated by the puncturing can be re-closed right away. Thus, the present invention is very practical and convenient.

Second, the present invention mainly combines the obtuse needle with the silicone layer. The present invention utilizes the obtuse needle that only generates a small puncturing hole. After the silicone layer is punctured, only one tiny point appears on the silicone layer. The tiny point will be sealed instantly and the artificial blood vessel is ready for re-use. Thus, the artificial blood vessel in accordance with the present invention has a prolonged lifetime and is more durable. It can also effectively prevent the blood from flowing into the artificial blood vessel and can prolong the lifetime of the artificial blood vessel.

Third, the present invention utilizes a silicone layer covered and mounted on the injection section of the artificial blood vessel body layer to carry out the thicker and enhanced injection section of the artificial blood vessel. Thus, the artificial blood vessel can be mounted in the skin layer tightly and effectively prevent the blood from flowing into the artificial blood vessel.

Although the invention has been explained in relation to its preferred embodiment, it is to be understood that many other possible modifications and variations can be made without departing from the spirit and scope of the invention as hereinafter claimed.

1. An artificial blood vessel, comprising:
an artificial blood vessel body layer comprising at least one injection section; and
a silicone layer covered and mounted on an outer surface of each of the at least one injection section.

2. The vessel of claim 1, wherein the number of the at least one injection section is two.

3. The vessel of claim 1, further comprising an obtuse needle.

4. The vessel of claim 3, wherein the obtuse needle comprises a bent injection end.

5. The vessel of claim 1, wherein the artificial blood vessel body layer is formed by injection-molding.

6. The vessel of claim 1, wherein the artificial blood vessel body layer and the silicone layer are formed integrally by injection-molding.
